(19) United States
(12) Patent Application Publication
(10) Pub. No.: US 2011/0158996 A1
(43) Pub. Date: Jun. 30, 2011

(54) VON WILLEBRAND FACTOR SPECIFIC BINDERS AND METHODS OF USE THEREFOR

(75) Inventors: Josefin-Beate Holz, Gent (BE); Karen Silence, Overijse (BE); Hans Ulrichts, Kortrijk (BE); Stefan De Buck, Magden (CH); Jozef Bartunek, Gent (BE); Robert Klamroth, Berlin (DE)

(73) Assignee: Ablynx N.V., Zwijnaarde (BE)

(21) Appl. No.: 12/922,748

(22) PCT Filed: Mar. 23, 2009
(86) PCT No.: PCT/EP2009/053385

§ 371 Date: Feb. 22, 2011

Related U.S. Application Data
(60) Provisional application No. 61/038,507, filed on Mar. 21, 2008.
(60) Provisional application No. 61/044,227, filed on Apr. 11, 2008.
(60) Provisional application No. 61/111,964, filed on Nov. 6, 2008.

(51) Int. Cl. A61K 39/395; G01N 33/68; A61P 7/02

(52) U.S. Cl. 424/135.1; 436/501

(57) ABSTRACT

The invention provides new uses for specific binders to the Al domain of the von Willebrand Factor (vWF), in particular the use in patients with stable angina undergoing elective percutaneous coronary intervention. Furthermore, dosing schedules and use of suitable assays such as RIPA and RICO in the particular disease settings are provided.

The invention provides new uses for specific binders to the A1 domain of the von Willebrand Factor (vWF), in particular the use in patients with stable angina undergoing elective percutaneous coronary intervention. Furthermore, dosing schedules and use of suitable assays such as Ristocetin-induced platelet aggregation (RIPA) and ristocetin cofactor activity (RICO) in the particular disease settings are provided.

### BACKGROUND OF THE INVENTION

Platelet aggregation is an essential event in the formation of blood clots. Under normal circumstances, blood clots serve to prevent the escape of blood cells from the vascular system. However, during certain disease states, clots can restrict or totally occlude blood flow resulting in cellular necrosis. For example, platelet aggregation and subsequent thrombosis at the site of an atherosclerotic plaque is an important causative factor in the genesis of conditions such as angina, acute myocardial infarction, and restenosis following successful thrombolysis and angioplasty. Current strategies to prevent thrombosis during and after angioplasty include the use of inhibitors of the platelet glycoprotein (GP)IIb/IIIa receptor (abciximab, ReoPro®) and inhibitors of the platelet P2Y12 receptor (clopidogrel, Plavix®). The most prominent risk of the currently used anti-thrombotic agents is an elevated bleeding diathesis or apparent bleeding. Hence, there is a clear medical need for improved anti-thrombotic agents with a superior clinical safety profile, in particular for anti-thrombotic agents with a decreased bleeding risk. Furthermore, Aspirin® (acetylsalicylic acid) and clopidogrel have emerged as critical therapies in the treatment of cardiovascular disease. Despite their efficacy, patients on these medications continue to suffer complications. Aspirin® (acetylsalicylic acid) and clopidogrel resistance is an emerging clinical observation with potentially severe consequences such as recurrent myocardial infarction, stroke, or death (Wang T H, Bhatt D L, Topol E J. Aspirin and clopidogrel resistance, an emerging clinical entity, European Heart Journal 2006:27, 647-54). Therefore, an increasing number of patients with resistances to the current anti-platelet regimens consisting of Aspirin®, and clopidogrel are in demand of novel therapeutics without cross-resistance and with novel mechanisms of action to maintain and support the treatment benefits in patients with acute coronary disease. Recent investigations in the early platelet activation cascade identified vWF as a key player in the initial steps of platelet adhesion to the vessel wall and subsequent thrombus formation in coronary arteries (Conway DSG. Prognostic Value of Plasma von Willebrand Factor and Soluble P-Selectin as Indices of Endothelial Damage and Platelet Activation in 994 Patients With Nonvalvular Atrial Fibrillation. Circulation 2003; 107; 3141-5). If an injury occurs in a vessel wall (plaque rupture or stent placing during a percutaneous coronary intervention procedure) subendothelial collagen becomes exposed and attracts platelets to form a thrombus. In vessels with high shear rates (e.g. coronary arteries), vWF binds to subendothelial collagen via its A3 domain, after which the A1 domain undergoes a structural change from a resting state to a conformation capable of interacting with the platelet receptor GPIb-IX-V. The reversible binding of GPIb-IX-V with collagen-bound vWF allows platelets to roll over the damaged area, which is then followed by a firm adhesion through the platelet collagen receptors (GPIa/IIa and GPVI) resulting in platelet activation. This leads to the conformational activation of the platelet GPIIb/IIIa receptor, fibrinogen binding, platelet aggregation, finally resulting in the formation of a thrombus.

Furthermore, it has been shown that the Nanobody® ALX-0081 (SEQ ID NO: 1) interrupts the binding between vWF and platelets, i.e. interrupts binding between the A1 domain of vWF and the glycoprotein 1b receptor (also GPIb) of the platelets, and that treatment with said a Nanobody® prevents thrombus formation in a baboon FOILS' model (see e.g. experiment 18 of WO2006/122825A2).

### SUMMARY OF THE INVENTION

It has now been found surprisingly that above mentioned polypeptide, ALX-0081 (SEQ ID NO: 1), can be administered in particular dosing regimens in humans. For example, ALX-0081 has been found to produce a pharmacodynamic effect, with a fast onset of action immediately at the end of dosing and maintains its efficacy for up to about 12 h. Additionally ALX-0081 (SEQ ID NO: 1) has been found to be well tolerated and safe in healthy male volunteers. These results indicate that ALX-0081 (SEQ ID NO: 1) and possibly similar specific A1 vWF binders may be suitable for acute treatment in patients with stable angina undergoing elective percutaneous coronary intervention (hereinafter also “PCI”). PCI is also commonly known as coronary angioplasty or simply angioplasty. PCI is a therapeutic procedure to treat the stenotic (narrowed) coronary arteries of the heart found in coronary heart disease. These stenotic segments are due to the build up of cholesterol-laden plaques that form due to atherosclerosis. PCI is usually performed by an invasive cardiologist.

Accordingly the present invention provides a method for the prevention of platelet aggregation and thrombus formation in patients, preferably humans, with stable angina undergoing elective percutaneous coronary intervention, wherein said prevention comprises administering an effective amount of a specific A1 vWF binder, e.g. ALX-0081 (SEQ ID NO: 1), to the patient.

The invention further provides use of a specific A1 vWF binder, e.g. ALX-0081 (SEQ ID NO: 1), in the preparation of a medicament for the prevention of platelet aggregation and thrombus formation in patients, preferably humans, with stable angina undergoing elective percutaneous coronary intervention.

The invention yet further provides use of a specific A1 vWF binder, e.g. ALX-0081 (SEQ ID NO: 1), to prevent platelet aggregation and thrombus formation in patients, preferably humans, with stable angina undergoing elective percutaneous coronary intervention and said patients are associated with other diseases or pathological conditions.

The present invention is particularly applicable to the safe prevention of platelet aggregation and thrombus formation in patients, i.e. the most prominent risk of the currently used non-vWF-specific anti-thrombotic agents, such as Plavix® (clopidogrel), Aspirin® (acetylsalicylic acid), Heparin® (heparin) and ReoPro® (abciximab), is an elevated bleeding diathesis or apparent bleeding.

A phase I double-blind, placebo-controlled, randomized parallel group, single ascending i.v. dose study was conducted in healthy male subjects (study ALX-0081-01/06) (see experimental part below). In this study, although bleeding time increases were observed with increased doses of ALX-0081, it can be noted that in all cases where the bleeding time was prolonged at 1 hour after start of infusion, bleeding stopped with a pressure bandage and tape and returned to below 10 min at the 12 hour time point at the latest—suggesting a superior (to existing anti-coagulants or anti-thrombotics) safety profile.

Preferably the invention is used for the acute treatment to prevent thrombus formation in patients with diseases and medical conditions in which existing anti-coagulants or anti-thrombotics such as Plavix® (clopidogrel), Aspirin® (acetylsalicylic acid), Heparin® (heparin) and ReoPro® (abciximab) cannot be used to inhibit platelet aggregation. For example, the invention may be used in the acute treatment to prevent thrombus formation in patients in need to inhibit platelet aggregation but that are resistant to the current anti-platelet regimens, e.g. as mentioned supra. Examples of such patients in need of anti-platelet regimens include patients with acute coronary syndromes undergoing PCI.

Furthermore, specific A1 vWF binders, e.g. ALX-0081, can be administered to an individual (e.g. a mammal such as a human) to prevent thrombosis as adjuvant therapy prior, during and/or post to a PCI.

More in particular, specific A1 vWF binders, e.g. ALX-0081, can be administered to an individual (e.g. a mammal such as a human) to prevent thrombosis as adjuvant therapy prior, during and/or post to an elective PCI.

More in particular, specific A1 vWF binders, e.g. ALX-0081, can be administered to an individual (e.g., a mammal such as a human) to prevent thrombosis as adjuvant therapy prior, during and/or post to an elective PCI in angina patients.

More in particular, specific A1 vWF binders, e.g. ALX-0081, can be administered to an individual (e.g. a mammal such as a human) to prevent thrombosis as adjuvant therapy prior, during and/or post to an elective PCI in stable angina patients.

### DETAILED DESCRIPTION OF THE INVENTION

The uses and methods of the present invention represent an improvement to existing therapy of coronary diseases in which specific A1 vWF binders are used to prevent or inhibit platelet aggregation or thrombus formation.

Thus in the present description the terms “treatment” or “treat” refer to both prophylactic or preventative treatment as well as curative or palliative treatment of inappropriate thrombus formation under high shear condition, e.g. they refer to an adjuvant treatment of stenotic coronary arteries or to prophylactic or preventative treatment in order to limit or completely reduce inappropriate thrombus formation under high shear condition at the stenotic coronary arteries, but the terms “treatment” or “treat” refer especially in the acute treatment setting in patients with stable angina undergoing elective PCI.

Thus in the present description the terms “prevent”, “preventing” and “prevention” (and the like) include, in addition to complete prevention, “reduce”, “reducing”, “reduction”, “inhibit”, “inhibiting” and “inhibition” of inappropriate thrombus formation under high shear condition.

Thus in particular embodiments, the invention provides:
a method for the prevention of thrombus formation under high shear condition in a patient with stable angina undergoing elective PCI which comprises administering an effective amount of a specific A1 vWF binder to the patient;
use of a specific A1 vWF binder in the preparation of a medicament for prevention of thrombus formation under high shear condition in patients with stable angina undergoing elective PCI; or
use of a specific A1 vWF binder as an agent for prevention of thrombus formation under high shear condition in patients with stable angina undergoing elective PCI.

The specific A1 vWF binders used in the present invention are typically those which prevent thrombus formation under high shear condition, in particular those which are indicated to have a safe application in patients with stable angina undergoing elective PCI, e.g. in patients in which the currently available anti-coagulants or anti-thrombotics are contra-indicated or lack sufficient efficacy and safety to fully prevent clinically relevant events.

Thus, for example, suitable agents of specific A1 vWF binders for use in the invention may include the compounds in Table 1 or a compound having 80% or more, more preferably 85% or more, most preferred 90%, 95%, 96%, 97%, 98%, 99% or more, amino acid sequence identity to a compound in Table 1 (see Definition section for “sequence identity”).

In another preferred selection, suitable agents of specific A1 vWF binders for use in the invention may include agents such as e.g. antibodies that cross-block or are cross-blocked by the compounds of Table 1 (see Definition section for “cross-blocked” and “cross-block”). In another preferred selection, suitable agents of specific A1 vWF binders for use according to the present invention are antibodies, preferably single variable domains, cross-blocking at least 50% of ALX-0081 (SEQ ID NO: 1) binding, more preferably at least 60%, more preferably at least 70%, even more preferably at least 80% of ALX-0081 binding. In another preferred selection, suitable agents of specific A1 vWF binders for use according to the present invention are antibodies, preferably single variable domains, cross-blocked at least 50% by ALX-0081 (SEQ ID NO: 1), more preferably at least 60%, more preferably at least 70%, even more preferably at least 80% by ALX-0081. Said cross-blocking or cross-blocked measurements are e.g. done by BiaCore measurements.

TABLE 1
Examples of specific A1 vWF binders

| | SEQ | |
|---|---|---|
| | ID | |
| Name | NO | Sequence |
| 12a2h1-3a- | 1 | EVQLVESGGGLVQPGGSLRLSCAASGRTFSY |
| 12a2h1 (ALX- | | NPMGWFRQAPGKGRELVAAISRTGGSTYYPD |
| 0081) | | SVEGRFTISRDNAKRMVYLQMNSLRAEDTAV |
| | | YYCAAAGVRAEDGRVRTLPSEYTFWGQGTQV |
| | | TVSSAAAEVQLVESGGGLVQPGGSLRLSCAA |
| | | SGRTFSYNPMGWFRQAPGKGRELVAAISRTG |
| | | GSTYYPDSVEGRFTISRDNAKRMVYLQMNSL |
| | | RAEDTAVYYCAAAGVRAEDGRVRTLPSEYTF |
| | | WGQGTQVTVSS |
| 12A-3a-12A2 | 2 | QVKLEESGGGLVQAGGALRLSCAASGRTFSY |
| | | NPMGWFRQAPGKERDLVAAISRTGGSTYYPD |
| | | SVEGRFTISRDNAKRMVYLQMNNLKPEDTAV |
| | | YYCAAAGVRAEDGRVRTLPSEYTFWGQGTQV |
| | | TVSSAAAEVQLVESGGGLVQAGGALRLSCAA |
| | | SGRTFSYNPMGWFRQAPGKERDLVAAISRTG |
| | | GSTYYPDSVEGRFTISRDNAKRMVYLQMNNL |
| | | KPEDTAVYYCAAAGVRAEDGRVRTLPSEYTF |
| | | WGQGTQVTVSS |
| 12A2-GS9-12A2 | 3 | QVKLEESGGGLVQAGGALRLSCAASGRTFSY |
| | | NPMGWFRQAPGKERDLVAAISRTGGSTYYPD |
| | | SVEGRFTISRDNAKRMVYLQMNNLKPEDTAV |
| | | YYCAAAGVRAEDGRVRTLPSEYTFWGQGTQV |
| | | TVSSGGGGSGGGSEVQLVESGGGLVQAGGAL |
| | | RLSCAASGRTFSYNPMGWFRQAPGKERDLVA |
| | | AISRTGGSTYYPDSVEGRFTISRDNAKRMVY |
| | | LQMNNLKPEDTAVYYCAAAGVRAEDGRVRTL |
| | | PSEYTFWGQGTQVTVSS |
| 12A2-GS30-12A2 | 4 | QVKLEESGGGLVQAGGALRLSCAASGRTFSY |
| | | NPMGWFRQAPGKERDLVAAISRTGGSTYYPD |
| | | SVEGRFTISRDNAKRMVYLQMNNLKPEDTAV |
| | | YYCAAAGVRAEDGRVRTLPSEYTFWGQGTQV |
| | | TVSSGGGGSGGGGSGGGGSGGGGSGGGGSGG |
| | | GGSEVQLVESGGGLVQAGGALRLSCAASGRT |
| | | FSYNPMGWFRQAPGKERDLVAAISRTGGSTY |
| | | YPDSVEGRFTISRDNAKRMVYLQMNNLKPEG |
| | | RAVYYCAAAGVRAEDGRVRTLPSEYTFWGQG |
| | | TQVTVSS |
| 12A5-3a-12A5 | 5 | AVQLVESGGGLVQPGGSLRLSCLASGRIFSI |
| | | GAMGMYRQAPGKQRELVATITSGGSTNYADP |
| | | VKGRFTISRDGPKNTVYLQMNSLKPEDTAVY |
| | | YCYANLKQGSYGYRFNDYWGQGTQVTVSSAA |
| | | AEVQLVESGGGLVQPGGSLRLSCLASGRIFS |
| | | IGAMGMYRQAPGKQRELVATITSGGSTNYAD |
| | | PVKGRFTISRDGPKNTVYLQMNSLKPEDTAV |
| | | YYCYANLKQGSYGYRFNDYWGQGTQVTVSS |
| 12A5-GS9-12A5 | 6 | AVQLVESGGGLVQPGGSLRLSCLASGRIFSI |
| | | GAMGMYRQAPGKQRELVATITSGGSTNYADP |
| | | VKGRFTISRDGPKNTVYLQMNSLKPEDTAVY |
| | | YCYANLKQGSYGYRFNDYWGQGTQVTVSSGG |
| | | GGSGGGSEVQLVESGGGLVQPGGSLRLSCLA |
| | | SGRIFSIGAMGMYRQAPGKQRELVATITSGG |
| | | STNYADPVKGRFTISRDGPKNTVYLQMNSLK |
| | | PEDTAVYYCYANLKQGSYGYRFNDYWGQGTQ |
| | | VTVSS |
| 12A5-GS30-12A5 | 7 | AVQLVESGGGLVQPGGSLRLSCLASGRIFSI |
| | | GAMGMYRQAPGKQRELVATITSGGSTNYADP |
| | | VKGRFTISRDGPKNTVYLQMNSLKPEDTAVY |
| | | YCYANLKQGSYGYRFNDYWGQGTQVTVSSGG |
| | | GGSGGGGSGGGGSGGGGSGGGGSGGGGSEVQ |
| | | LVESGGGLVQPGGSLRLSCLASGRIFSIGAM |
| | | GMYRQAPGKQRELVATITSGGSTNYADPVKG |
| | | RFTISRDGPKNTVYLQMNSLKPEDTAVYYCY |
| | | ANLKQGSYGYRFNDYWGQGTQVTVSS |
| 12B6-3a-12B6 | 8 | QVQLVESGGGLVQAGGALRLSCAASGRTGSY |
| | | NPMGWFRQAPGKERDVVAAISRTGGSTYYAR |
| | | SVEGRFTISRDNAKRMVYLQMNALKPEDTAV |
| | | YYCAAAGVRAEDGRVRTLPSEYNFWGQGTQV |
| | | TVSSAAAEVQLVESGGGLVQAGGALRLSCAA |
| | | SGRTFSYNPMGWFRQAPGKERDVVAAISRTG |
| | | GSTYYARSVEGRFTISRDNAKRMVYLQMNAL |
| | | KPEDTAVYYCAAAGVRAEDGRVRTLPSEYNF |
| | | WGQGTQVTVSS |
| 12B6-GS9-12B6 | 9 | QVQLVESGGGLVQAGGALRLSCAASGRTFSY |
| | | NPMGWFRQAPGKERDVVAAISRTGGSTYYAR |
| | | SVEGRFTISRDNAKRMVYLQMNALKPEDTAV |
| | | YYCAAAGVRAEDGRVRTLPSEYNFWGQGTQV |
| | | TVSSGGGGSGGGSEVQLVESGGGLVQAGGAL |
| | | RLSCAASGRTFSYNPMGWFRQAPGKERDVVA |
| | | AISRTGGSTYYARSVEGRFTISRDNAKRMVY |
| | | LQMNALKPEDTAVYYCAAAGVRAEDGRVRTL |
| | | PSEYNFWGQGTQVTVSS |
| 12B6-GS30-12B6 | 10 | QVQLVESGGGLVQAGGALRLSCAASGRTFSY |
| | | NPMGWFRQAPGKERDVVAAISRTGGSTYYAR |
| | | SVEGRFTISRDNAKRMVYLQNMALKPEDTAV |
| | | YYCAAAGVRAEDGRVRTLPSEYNFWGQGTQV |
| | | TVSSGGGGSGGGGSGGGGSGGGGSGGGGSGG |
| | | GGSEVQLVESGGGLVQAGGALRLSCAASGRT |
| | | FSYNPMGWFRQAPGKERDVVAAISRTGGSTY |
| | | YARSVEGRFTISRDNAKRMVYLQMNALKPED |
| | | TAVYYCAAAGVRAEDGRVRTLPSEYNFWGQG |
| | | TQVTVSS |
| 12A2H4-3a- | 11 | EVQLVESGGGLVQPGGSLRLSCAASGRRFSY |
| 12A2H4 | | NPMGWFRQAPGKGRELVAAISRTGGSRYYPD |
| | | SVEGRFTISRDNAKRSVYLQMNSLRAEDTAV |
| | | YYCAAAGVRAEDGRVRTLPSEYTFWGQGTQV |
| | | TVSSAAAEVQLVESGGGLVQPGGSLRLSCAA |
| | | SGRTFSYNPMGWFRQAPGKGRELVAAISRTG |
| | | GSTYYPDSVEGRFTISRDNAKRSVYLQMNSL |
| | | RAEDTAVYYCAAAGVRAEDGRVRTLPSEYTF |
| | | WGQGTQVTVSS |
| 12B6H2-3a- | 12 | EVQLVESGGGLVQPGGSLRLSCAASGRTFSY |
| 12B6H2 | | NPMGWFRQAPGKGREVVAAISRTGGSTYYAR |
| | | SVEGRFTISRDVAKRMVYLQMNSLRAEDTAV |
| | | YYCAAAGVRAEDGRVRTLPSEYNFWGQGTQV |
| | | TVSSAAAEVQLVESGGGLVQPGGSLRLSCAA |
| | | SGRTFSYNPMGWFRQAPGKGREVVAAISRTG |
| | | GSTYYARSVEGRFTISRDNAKRMVYLQMNSL |
| | | RAEDTAVYYCAAAGVRAEDGRVRTLPSEYNF |
| | | WGQGTQVTVSS |
| 12A2H1-GS9- | 13 | EVQLVESGGGLVQPGGSLRLSCAASGRTFSY |
| 12A2H1 | | NPMGWFRQAPGKGRELVAAISRTGGSTYYPD |
| | | SVEGRFTISRDNAKRMVYLQMNSLRAEDTAV |
| | | YYCAAAGVRAEDGRVRTLPSEYTFWGQGTQV |
| | | TVSSGGGGSGGGSEVQLVESGGGLVQPGGSL |
| | | RLSCAASGRTFSYNPMGWFRQAPGKGRELVA |
| | | AISRTGGSTYYPDSVEGRTTISRDNAKRMVY |
| | | LQMNSLRAEDTAVYYCAAAGVRAEDGRVRTL |
| | | PSEYTFWGQGTQVTVSS |
| 12A2H4-GS9- | 14 | EVQLVESGGGLVQPGGSLRLSCAASGRTFSY |
| 12A2H4 | | NPMGWFRQAPGKGRELVAAISRTGGSTYYPD |
| | | SVEGRFTISRDNAKRSVYLQMNSLRAEDTAV |
| | | YYCAAAGVRAEDGRVRTLPSEYTFWGQGTQV |
| | | TVSSGGGGSGGGSEVQLVESGGGLVQPGGSL |
| | | RLSCAASGRTFSYNPMGWFRQAPGKGRELVA |
| | | AISRTGGSTYYPDSVEGRFTISRDNAKRSVY |
| | | LQMNSLRAEDTAVYYCAAAGVRAEDGRVRTL |
| | | PSEYTFWGQGTQVTVSS |
| 12B6H2-GS9- | 15 | EVQLVESGGGLVQPGGSLRLSCAASGRTFSY |
| 12B6H2 | | NPMGWFRQAPGKGREVVAAISRTGGSTYYAR |
| | | SVEGRFTISRDNAKRMVYLQMNSLRAEDTAV |
| | | YYCAAAGVRAEDGRVRTLPSEYNFWGQGTQV |
| | | TVSSGGGGSGGGSEVQLVESGGGLVQPGGSL |
| | | RLSCAASGRTFSYNPMGWFRQAPGKGREVVA |
| | | AISRTGGSTYYARSVEGRFTISRDNAKRMVY |
| | | LQMNSLRAEDTAVYYCAAAGVRAEDGRVRTL |
| | | PSEYNFWGQGTQVTVSS |
| 12A2H1-GS30- | 16 | EVQLVESGGGLVQPGGSLRLSCAASGRTFSY |
| 12A2H1 | | NPMGWFRQAPGKGRELVAAISRTGGSTYYPD |
| | | SVEGRFTISRDNAKRMVYLQMNSLRAEDTAV |
| | | YYCAAAGVRAEDGRVRTLPSEYTPWGQGTQV |
| | | TVSSGGGGSGGGGSGGGGSGGGGSGGGGSGG |
| | | GGSEVQLVESGGGLVQPGGSLRLSCAASGRT |
| | | FSYNPMGWFRQAPGKGRELVAAISRTGGSTY |
| | | YPDSVEGRFTISRDNAKRMVYLQMNSLRAED |
| | | TAVYYCAAAGVRAEDGRVRTLPSEYTFWGQG |
| | | TQVTVSS |
| 12A2H4-GS30- | 17 | EVQLVESGGGLVQPGGSLRLSCAASGRTFSY |
| 12A2H4 | | NPMGWFRQAPGKGRELVAAISRTGGSTYYPD |
| | | SVEGRFTISRDNAKRSVYLQMNSLRAEDTAV |
| | | YYCAAAGVRAEDGRVRTLPSEYTFWGQGTQV |
| | | TVSSGGGGSGGGGSGGGGSGGGGSGGGGSGG |
| | | GGSEVQLVESGGGLVQPGGSLRLSCAASGRT |
| | | FSYNPMGWFRQAPGKGRELVAAISRTGGSTY |
| | | YPDSVEGRFTISRDNAKRSVYLQMNSLRAED |
| | | TAVYYCAAAGVRAEDGRVRTLPSEYTFWGQG |
| | | TQVTVSS |
| 12B6H2-GS30- | 18 | EVQLVESGGGLVQPGGSLRLSCAASGRTFSY |
| 12B6H2 | | NPMGWFRQAPGKGREVVAAISRTGGSTYYAR |
| | | SVEGRFTISRDNAKRMVYLQMNSLRAEDTAV |
| | | YYCAAAGVRAEDGRVRTLPSEYNFWGQGTQV |
| | | TVSSGGGGSGGGGSGGGGSGGGGSGGGGSGG |
| | | GGSEVQLVESGGGLVQPGGSLRLSCAASGRT |
| | | FSYNPMGWFRQAPGKGREVVAAISRTGGSTY |
| | | YARSVEGRFTISRDNAKRMVYLQMNSLRAED |
| | | TAVYYCAAAGVRAEDGRVRTLPSEYNFWGQG |
| | | TQVTVSS |

Preferably the specific A1 vWF binders for use in the invention are the 12a2h1-like compounds. For the purposes of the present description a 12a2h1-like compound is a compound which comprises 12a2h1 (i.e. SEQ ID NO: 19) or a compound having 80% or more, more preferably 85% or more, most preferred 90%, 95%, 96%, 97%, 98%, 99% or more, amino acid sequence identity to 12a2h1 (SEQ ID NO: 19):

| | | |
|---|---|---|
| 12a2h1 | 19 | EVQLVESGGGLVQPGGSLRLSCAASGRTFSYNPMGW |
| | | FRQAPGKGRELVAAISRTGGSTYYPDSVEGRFTISR |
| | | DNAKRMVYLQMNSLRAEDTAVYYCAAAGVRAEDGRV |
| | | RTLPSEYTFWGQGTQVTVSS |

A particularly preferred specific A1 vWF binder is ALX-0081 (SEQ ID NO: 1

All the specific A1 vWF binders mentioned above are well known from the literature. This includes their manufacture (see in particular e.g. WO 2006/122825 but also WO 2004/062551). For example, ALX-0081 is prepared as described e.g. in WO 2006/122825.

The specific A1 vWF binders (hereinafter referred to as the Agents of the Invention) may be used in the form of a polypeptide concentrate or ready-to-use solution (hereinafter also referred to as “pharmaceutical composition of the invention”). For example, the Agents of the Invention can be used in a pharmaceutical composition comprising a buffer (such as e.g. citrate, histidine, Tris, PBS, d-PBS), a tonicifier (such as e.g. mannitol, glycine or sodium chloride) and a surfactant (such as e.g. Polysorbate 80 or Polysorbate 20). Additionally, osmolytes and preservatives may be added. The Agents of the Invention may be in a small-volume, high-dose solution such as e.g. in an amount of from 1 mg agent per ml solution up to 50 mg agent per ml solution. Other concentrations such as e.g. 2, 3, 4, 5, 6, 7, 8, 9, 10, 11, 12, 13, 14, 15, 16, 17, 18, 19, 20, 25, 30, 35, 40, or 45 are also feasible. A preferred pharmaceutical formulation for ALX-0081 comprises between 1 to 20 mg, e.g. 5 to 10 mg, ALX-0081 per ml solution that comprises a buffer, a tonicifier and a surfactant. A more preferred pharmaceutical composition comprises between 1 to 20 mg ALX-0081 per ml solution that consists of a buffer, e.g. d-PBS, a tonicifier, e.g. glycine, and a surfactant, e.g. Polysorbate 80. An even more preferred pharmaceutical composition comprises 5 (+/−1) mg/ml ALX-0081, suitable d-PBS buffer; suitable amount of glycine; and a suitable amount of Polysorbate 80 pH 7.1. A most preferred pharmaceutical composition comprises 5 (+/−1) mg/ml ALX-0081, 0.137 M NaCl, 3.7 mM KH₂PO₄, 9.8 mM Na₂HPO₄×2H₂O, 2.7 KCl, 0.2 M glycine. 0.02% (volume %) Polysorbate 80 pH 7.1. Said compositions may be in the form of a concentrate and thus e.g. the dose applied to a patient in need thereof may be adopted by diluting the concentrate to the desired dose (see e.g. experimental part for suitable doses).

The Agents of the Invention (the specific A1 vWF binders) are preferably used in the form of pharmaceutical compositions that contain a therapeutically effective amount of active ingredient optionally together with or in admixture with inorganic or organic, solid or liquid, pharmaceutically acceptable carriers which are suitable for administration.

The pharmaceutical compositions may be, for example, compositions for parenteral, such as intravenous or subcutaneous administration, or compositions for transdermal administration (e.g. passive or iontophoretic).

Preferably, the pharmaceutical compositions are adapted to parenteral (especially intravenous, intra-arterial or transdermal) administration. Intravenous administration is considered to be of particular importance. Preferably the specific A1 vWF binder is in the form of a parenteral form, most preferably an intravenous form.

The particular mode of administration and the dosage may be selected by the attending physician taking into account the particulars of the patient, especially age, weight, life style, activity level, and general medical condition as appropriate. More specifically, ALX-0081 is administered intravenously in a 6 h dose interval. Even more preferably, ALX-0081, is administered intravenously in a 6 h dose interval upon consideration of the aggregation activity, e.g. measured by RIPA, ristocetin induced platelet aggregation—(Favaloro E J. Clin Haematol 2001; 14: 299-319.) and/or Ristocetin Cofactor Platelet Agglutination Assay—(Howard M A, Firkin B G. Ristocetin—a new tool in the investigation of platelet aggregation. Thrombosis et Diathesis Haemorrhagica 1971; 26: 362-9). For example, a further dose is not administered if the aggregation activity is estimated to stay below 10% measured by RIPA or stay below 20% measured by RICO for the next 6 hours (Clinically relevant inhibition).

However, in general the dosage of the Agents of the Invention may depend on various factors, such as effectiveness and duration of action of the active ingredient, warm-blooded species, and/or sex, age, weight and individual condition of the warm-blooded animal.

Normally the dosage is such that a single dose of a specific A1 vWF binder, e.g. is estimated based on in vitro results, or e.g. based on results from a dose escalating study to test subchronic toxicity in cynomolgus monkeys. Based on such a preclinical data set, a starting and subsequent escalating dose for a specific A1 vWF binder can be determined. E.g. a dose may be from 0.5-50.0 mg, especially 1-30.0 mg, and is administered to a warm-blooded animal weighing approximately 75 (+/−30) kg (but can be different as well to this norm). If desired, this dose may also be taken in several, optionally equal, partial doses (“mg” means mg drug per mammal—including human—to be treated). For the purposes of the Agent of the invention, it is surprising to find that doses need not be adjusted to weight and thus this is another advantage of the invention.

The dose mentioned above—either administered as a single dose (which is one embodiment) or in several partial doses—may be repeated, as mentioned above for example once every six hours, once every 12 hours, or once daily. In other words, the pharmaceutical compositions may be administered in regimens ranging from continuous 6 hourly therapy to longer interval dosing therapy.

Preferably, the specific A1 vWF binders are administered in doses which are in the same order of magnitude as those used in the adjunct treatment in patients in need for PCI as herein suggested for ALX-0081. For example, for the preferred 12a2h1-containing specific A1 vWF binders, e.g. ALX-0081 and functional variants thereof, doses of specific A1 vWF binders in the range from about 0.5 to about 12 mg, preferably from about 2 to about 12 mg, more preferably from 4 to about 8 mg, may be used for acute treatment in human patients.

Formulations in single dose unit form contain preferably from about 1 to about 5 mg/ml and formulations not in single dose unit form contain preferably from also about 1 to about 5 mg/ml of the active ingredient.

Pharmaceutical preparations for parenteral administration are, for example, those in dosage unit forms, such as ampoules. They are prepared in a manner known per se, for example by means of conventional mixing, dissolving or lyophilising processes.

Parenteral formulations are especially injectable fluids that are effective in various manners, such as at site of PCI, intra-arterially, intramuscularly, intraperitoneally, intranasally, intradermally, subcutaneously or preferably intravenously. Such fluids are preferably isotonic aqueous solutions or suspensions which can be prepared before use, for example from lyophilised preparations or concentrate which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers.

Suitable formulations for transdermal application include an effective amount of the active ingredient with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient of the skin of the host at a controlled and predetermined rate over a prolonged period of time, and means to secure the device to the skin.

### DEFINITIONS

a) For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e. as an “amino acid difference” as defined herein.

Alternatively, the degree of sequence identity between two amino acid sequences may be calculated using a known computer algorithm, such as those mentioned above for determining the degree of sequence identity for nucleotide sequences, again using standard settings.

Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-3 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein. Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (h) polar, negatively charged residues and their (uncharged) amides: Asp, Asn, Glu and Gln; (e) polar, positively charged residues: His, Arg and Lys; (d) large aliphatic, nonpolar residues: Met, Leu, Ile, Val and Cys; and (e) aromatic residues: Phe, Tyr and Trp. Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu. Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Natl. Acad. Sci. USA 81: 140-144, 1984; Kyte & Doolittle; J. Malec. Biol. 157: 105-132, 1981, and Goldman et al., Ann. Rev. Biophys. Chem. 15: 321-353, 1986, all incorporated herein in their entirety by reference. Information on the primary, secondary and tertiary structure of Nanobodies® is given in the description herein and in the general background art cited above. Also, for this purpose, the crystal structure of a V_{HH} domain from a llama is for example given by Desmyter et al., Nature Structural Biology, Vol. 3, 9, 803 (1996); Spinelli et al., Natural Structural Biology (1996); 3, 752-757; and Decanniere et al., Structure, Vol. 7, 4, 361 (1999). Further information about some of the amino acid residues that in conventional V_{H} domains form the V_{H}/V_{L} interface and potential camelizing substitutions on these positions can be found in the prior art cited above.

b) The terms “cross-block”, “cross-blocked” and “cross-blocking” are used interchangeably herein to mean the ability of an amino acid sequence or other binding agents (such as a polypeptide of the invention) to interfere with the binding of other amino acid sequences or binding agents of the invention to a given target. The extent to which an amino acid sequence or other binding agents of the invention is able to interfere with the binding of another to the A1 domain of vWF, and therefore whether it can be said to cross-block according to the invention, can be determined using competition binding assays. One particularly suitable quantitative assay uses a Biacore machine which can measure the extent of interactions using surface plasmon resonance technology. Another suitable quantitative cross-blocking assay uses an ELISA-based approach to measure competition between amino acid sequence or another binding agents in terms of their binding to the target. The following generally describes a suitable Biacore assay for determining whether an amino acid sequence or other binding agent cross-blocks or is capable of cross-blocking according to the invention. It will be appreciated that the assay can be used with any of the amino acid sequence or other binding agents described herein. The Biacore machine (for example the Biacore 3000) is operated in line with the manufacturer's recommendations. Thus in one cross-blocking assay, the target protein is coupled to a CM5 Biacore chip using standard amine coupling chemistry to generate a surface that is coated with the target. Typically 200-800 resonance units of the target would be coupled to the chip (an amount that gives easily measurable levels of binding but that is readily saturable by the concentrations of test reagent being used). Two test amino acid sequences (termed A* and B*) to be assessed for their ability to cross-block each other are mixed at a one to one molar ratio of binding sites in a suitable buffer to create the test mixture. When calculating the concentrations on a binding site basis the molecular weight of an amino acid sequence is assumed to be the total molecular weight of the amino acid sequence divided by the number of target binding sites on that amino acid sequence. The concentration of each amino acid sequence in the test mix should be high enough to readily saturate the binding sites for that amino acid sequence on the target molecules captured on the Biacore chip. The amino acid sequences in the mixture are at the same molar concentration (on a binding basis) and that concentration would typically be between 1.00 and 1.5 micromolar (on a binding site basis). Separate solutions containing A* alone and B* alone are also prepared. A* and B* in these solutions should be in the same buffer and at the same concentration as in the test mix. The test mixture is passed over the target-coated Biacore chip and the total amount of binding recorded. The chip is then treated in such a way as to remove the bound amino acid sequences without damaging the chip-bound target. Typically this is done by treating the chip with 30 mM HCl for 60 seconds. The solution of A* alone is then passed over the target-coated surface and the amount of binding recorded. The chip is again treated to remove all of the bound amino acid sequences without damaging the chip-bound target. The solution of B* alone is then passed over the target-coated surface and the amount of binding recorded. The maximum theoretical binding of the mixture of A* and B* is next calculated, and is the sum of the binding of each amino acid sequence when passed over the target surface alone. If the actual recorded binding of the mixture is less than this theoretical maximum then the two amino acid sequences are cross-blocking each other. Thus, in general, a cross-blocking amino acid sequence or other binding agent according to the invention is one which will bind to the target in the above Biacore cross-blocking assay such that during the assay and in the presence of a second amino acid sequence or other binding agent of the invention the recorded binding is between 80% and 0.1% (e.g. 80% to 4%) of the maximum theoretical binding, specifically between 75% and 0.1% (e.g. 75% to 4%) of the maximum theoretical binding, and more specifically between 70% and 0.1% (e.g. 70% to 4%) of maximum theoretical binding (as just defined above) of the two amino acid sequences or binding agents in combination. The Biacore assay described above is a primary assay used to determine if amino acid sequences or other binding agents cross-block each other according to the invention. On rare occasions particular amino acid sequences or other binding agents may not bind to target coupled via amine chemistry to a CM5 Biacore chip (this usually occurs when the relevant binding site on target is masked or destroyed by the coupling to the chip). In such cases cross-blocking can be determined using a tagged version of e.g., vWF or a fragment thereof containing at least the A1 domain, for example a N-teirminal His-tagged version (R 8 D Systems, Minneapolis, Mid, USA; 2005 cat #1406-ST-025). In this particular format, an anti-His amino acid sequence would be coupled to the Biacore chip and then the His-tagged target would be passed over the surface of the chip and captured by the anti-His amino acid sequence. The cross blocking analysis would be carried out essentially as described above, except that after each chip regeneration cycle, new His-tagged target would be loaded back onto the anti-His amino acid sequence coated surface. In addition to the example given using N-terminal. His-tagged vWF or fragment thereof containing at least the A1 domain, C-terminal His-tagged target could alternatively be used. Furthermore, various other tags and tag binding protein combinations that are known in the art could be used for such a cross-blocking analysis (e.g. HA tag with anti-HA antibodies; FLAG tag with anti-FLAG antibodies; biotin tag with streptavidin). The following generally describes an ELISA assay for determining whether an amino acid sequence or other binding agent directed against a target cross-blocks or is capable of cross-blocking as defined herein. It will be appreciated that the assay can be used with any of the amino acid sequences (or other binding agents such as polypeptides of the invention) described herein. The general principal of the assay is to have an amino acid sequence or binding agent that is directed against the target coated onto the wells of an ELISA plate. An excess amount of a second, potentially cross-blocking, anti-target amino acid sequence is added in solution (i.e. not bound to the ELISA plate). A limited amount of the target is then added to the wells. The coated amino acid sequence and the amino acid sequence in solution compete for binding of the limited number of target molecules. The plate is washed to remove excess target that has not been bound by the coated amino acid sequence and to also remove the second, solution phase amino acid sequence as well as any complexes formed between the second, solution phase amino acid sequence and target. The amount of bound target is then measured using a reagent that is appropriate to detect the target. An amino acid sequence in solution that is able to cross-block the coated amino acid sequence will be able to cause a decrease in the number of target molecules that the coated amino acid sequence can bind relative to the number of target molecules that the coated amino acid sequence can bind in the absence of the second, solution phase, amino acid sequence. In the instance where the first amino acid sequence, e.g. an Ab-X, is chosen to be the immobilized amino acid sequence, it is coated onto the wells of the ELISA plate, after which the plates are blocked with a suitable blocking solution to minimize non-specific binding of reagents that are subsequently added. An excess amount of the second amino acid sequence, i.e. Ab-Y, is then added to the ELISA plate such that the moles of Ab-Y [target] binding sites per well are at least 10 fold higher than the moles of Ab-X [target] binding sites that were used, per well, during the coating of the ELISA plate. [target] is then added such that the moles of [target] added per well are at least 25-fold lower than the moles of Ab-X [target] binding sites that were used for coating each well. Following a suitable incubation period the ELISA plate is washed and a reagent for detecting the target is added to measure the amount of target specifically bound by the coated anti-[target] amino acid sequence (in this case Ab-X). The background signal for the assay is defined as the signal obtained in wells with the coated amino acid sequence (in this case Ab-X), second solution phase amino acid sequence (in this case Ab-Y), [target] buffer only (i.e. no target) and target detection reagents. The positive control signal for the assay is defined as the signal obtained in wells with the coated amino acid sequence (in this case Ah-X), second solution phase amino acid sequence buffer only (i.e. no second solution phase amino acid sequence), target and target detection reagents. The ELISA assay may be run in such a manner so as to have the positive control signal be at least 6 times the background signal. To avoid any artefacts (e.g. significantly different affinities between Ab-X and Ab-Y for [target]) resulting from the choice of which amino acid sequence to use as the coating amino acid sequence and which to use as the second (competitor) amino acid sequence, the cross-blocking assay may to be run in two formats: 1) format 1 is where Ab-X is the amino acid sequence that is coated onto the ELISA plate and Ab-Y is the competitor amino acid sequence that is in solution and 2) format 2 is where Ab-Y is the amino acid sequence that is coated onto the ELISA plate and Ab-X is the competitor amino acid sequence that is in solution. Ab-X and Ab-Y are defined as cross-blocking if, either in format 1 or in format 2, the solution phase anti-target amino acid sequence is able to cause a reduction of between 60% and 100%, specifically between 70% and 100%, and more specifically between 80% and 100%, of the target detection signal {i.e. the amount of target bound by the coated amino acid sequence) as compared to the target detection signal obtained in the absence of the solution phase anti-target amino acid sequence (i.e. the positive control wells).

c) The term “specific” refers to the number of different types of antigens or antigenic determinants to which a particular antigen-binding molecule or antigen-binding protein (such as a Nanobody® or a polypeptide of the invention) molecule can bind. The specificity of an antigen-binding protein can be determined based on affinity and/or avidity. The affinity, represented by the equilibrium constant for the dissociation of an antigen with an antigen-binding protein (K_{D}), is a measure for the binding strength between an antigenic determinant and an antigen-binding site on the antigen-binding protein: the lesser the value of the K_{D}, the stronger the binding strength between an antigenic determinant and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as a Nanobody® or polypeptide of the invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the amino acid sequences, Nanobodies® and/or polypeptides of the invention) will bind to their antigen with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10 to 10¹² liter/moles). Any K_{D} value greater than 10⁴ mol/liter (or any K_{A} value lower than 10⁴ M⁻¹) liters/mol is generally considered to indicate non-specific binding. Preferably, a monovalent immunoglobulin sequence of the invention will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein. The dissociation constant may be the actual or apparent dissociation constant, as will be clear to the skilled person. Methods for determining the dissociation constant will be clear to the skilled person, and for example include the techniques mentioned herein. In this respect, it will also be clear that it may not be possible to measure dissociation constants of more then 10⁻⁴ moles/liter or 10⁻³ moles/liter (e.g., of 10⁻² moles/liter). Optionally, as will also be clear to the skilled person, the (actual or apparent) dissociation constant may be calculated on the basis of the (actual or apparent) association constant (K_{A}), by means of the relationship [K_{D}=1/K_{A}].

The affinity denotes the strength or stability of a molecular interaction. The affinity is commonly given as by the K_{D}, or dissociation constant, which has units of mol/liter (or M). The affinity can also be expressed as an association constant, K_{A}, which equals 1/K_{D} and has units of (mol/liter)⁻¹ (or M⁻¹). In the present specification, the stability of the interaction between two molecules (such as an amino acid sequence, Nanobody or polypeptide of the invention and its intended target) will mainly be expressed in terms of the K_{D} value of their interaction; it being clear to the skilled person that in view of the relation K_{A}=1/K_{D}, specifying the strength of molecular interaction by its K_{D}) value can also be used to calculate the corresponding K_{A} value. The K_{D}-value characterizes the strength of a molecular interaction also in a thermodynamic sense as it is related to the free energy (DG) of binding by the well known relation DG=RT.ln(K_{D}) (equivalently DG=−RT.ln(K_{A})), where R equals the gas constant, T equals the absolute temperature and In denotes the natural logarithm. The K_{D} for biological interactions which are considered meaningful (e.g. specific) are typically in the range of 10⁻¹⁰M (0.1 nM) to 10⁻⁵M (10000 nM). The stronger an interaction is, the lower is its K_{D}. The K_{D} can also be expressed as the ratio of the dissociation rate constant of a complex, denoted as k_{off}, to the rate of its association, denoted kₒₙ (so that K_{D}=k_{off}/kₒₙ and K_{A}=kₒₙ/k_{off}). The off-rate k_{off} has units s⁻¹ (where s is the SI unit notation of second). The on-rate kₒₙ has units M⁻¹s⁻¹. The on-rate may vary between 10² M⁻¹s⁻¹ to about 10⁷ M⁻¹s⁻¹, approaching the diffusion-limited association rate constant for bimolecular interactions. The off-rate is related to the half-life of a given molecular interaction by the relation t_{1/2} ln(2)/k_{off}. The off-rate may vary between 10⁻⁶ s⁻¹ (near irreversible complex with a t_{1/2} of multiple days) to 1 s⁻¹ (t_{1/2}=0.69 s).

The affinity of a molecular interaction between two molecules can be measured via different techniques known per se, such as the well known surface plasmon resonance (SPR) biosensor technique (see for example Ober et al., Intern. Immunology, 13, 1551-1559, 2001) where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BIACORE instruments.

It will also be clear to the skilled person that the measured K_{D} may correspond to the apparent K_{D} if the measuring process somehow influences the intrinsic binding affinity of the implied molecules for example by artifacts related to the coating on the biosensor of one molecule. Also, an apparent K_{D} may be measured if one molecule contains more than one recognition sites for the other molecule. In such situation the measured affinity may be affected by the avidity of the interaction by the two molecules.

Another approach that may be used to assess affinity is the 2-step ELISA (Enzyme-Linked Immunosorbent Assay) procedure of Friguet et al. (J. Immunol. Methods, 77, 305-19, 1985). This method establishes a solution phase binding equilibrium measurement and avoids possible artefacts relating to adsorption of one of the molecules on a support such as plastic. However, the accurate measurement of K_{D} may be quite labor-intensive and as consequence, often apparent K_{D} values are determined to assess the binding strength of two molecules. It should be noted that as long all measurements are made in a consistent way (e.g. keeping the assay conditions unchanged) apparent K_{D} measurements can be used as an approximation of the true K_{D} and hence in the present document K_{D} and apparent K_{D} should be treated with equal importance or relevance.

Finally, it should be noted that in many situations the experienced scientist may judge it to be convenient to determine the binding affinity relative to some reference molecule. For example, to assess the binding strength between molecules A and B, one may e.g. use a reference molecule C that is known to bind to B and that is suitably labelled with a fluorophore or chromophore group or other chemical moiety, such as biotin for easy detection in an ELISA or FACS (Fluorescent activated cell sorting) or other format (the fluorophore for fluorescence detection, the chromophore for light absorption detection, the biotin for streptavidin-mediated ELISA detection). Typically, the reference molecule C is kept at a fixed concentration and the concentration of A is varied for a given concentration or amount of B. As a result an IC₅₀ value is obtained corresponding to the concentration of A at which the signal measured for C in absence of A is halved. Provided K_{D ref}, the K_{D} of the reference molecule, is known, as well as the total concentration c_{ref} of the reference molecule, the apparent K_{D} for the interaction A-B can be obtained from following formula: K_{D}=IC₅₀/(1+c_{ref}/K_{D ref}). Note that if c_{ref}<<K_{D ref}, K_{D}≈IC₅₀. Provided the measurement of the IC₅₀ is performed in a consistent way (e.g. keeping c_{ref} fixed) for the binders that are compared, the strength or stability of a molecular interaction can be assessed by the IC₅₀ and this measurement is judged as equivalent to K_{D} or to apparent K_{D} throughout this text.

The following Experimental Part illustrates the invention described hereinbefore.

### Experimental Part

### Example 1

### Double-Blind, Placebo-Controlled, Randomized Parallel Group, Single Ascending i.v. Dose Study was Conducted in Healthy Male Subjects

A phase I double-blind, placebo-controlled, randomized parallel group, single ascending i.v. dose study was conducted in healthy male subjects. This study was designed to assess the safety, tolerability, PK and PD of ALX-0081 (SEQ ID NO: 1). The starting dose of study medication was i.v. 500 μg ALX-0081 or placebo (dose level 1) followed by 2-fold, 4-fold, 8-fold, 16-fold, and 24-fold of the starting dose in dose levels 2-6, respectively. The desired dose of ALX-0081 is provided by adding the corresponding amount (dose levels 1 to 6) of ALX-0081 drug product (see Table E-1) to water for injection. A total of 100 mL solution for infusion was prepared, whereas only 50 mL solution for infusion was administered per i.v. infusion over 60 minutes via an infusion pump.

TABLE E-1
ALX-0081 drug product

5 mg/ml ALX-0081
0.137 M NaCl
3.7 mM KH₂PO₄
9.8 mM Na₂HPO₄ × 2H₂O
2.7 mM KCl
0.2M Glycine
0.02% (volume %) Tween-80 (Polysorbate 80) pH 7.1

The final analysis of this phase I study based on the data of six dosing cohorts with n=6 subjects per cohort (n=3 ALX-0081 and n=3 placebo) for cohorts 1-5 and n=10 subjects (n=6 ALX-0081 and n=4 placebo) in cohort 6 allows the following conclusions:
A single, fixed dose of ALX-0081, administered as i.v. infusion over 1 hour was safe and well tolerated.
ALX-0081 displayed non-linear PK properties, following a 2 compartment model.
RIPA was analyzed as marker for PD effect with full inhibition (defined as measured levels dropping <10%) observed at ALX-0081 concentrations of ˜400 ng/mL. All subjects dosed ≧2 mg achieved full RIPA inhibition at 1 h post-dosing for maximum of 12 h.
The extent and duration of RIPA inhibition was in good correlation with the administered dose of ALX-0081 and suggests the suitability of this biomarker to assess the effectiveness of ALX-0081, while Template Bleeding Time did not stringently correlate with the other pharmacodynamic or pharmacological effects of ALX-0081.

| | | | | | vWF max |
|---|---|---|---|---|---|
| | | | | FVIII max | reduction |
| | | | RIPA < 10% | reduction from | from baseline |
| Cohort | | # Subjects | (duration) | baseline [%] | [%] |
| 0.5 | mg | 3 | 0 | 22% | 26% |
| 1 | mg | 3 | 1 (2 h) | 24% | 35% |
| 2 | mg | 3 | 3 (3-4 h) | 30% | 36% |
| 4 | mg | 3 | 3 (4-6 h) | 43% | 50% |
| 8 | mg | 3 | 3 (4-8 h) | 37% | 40% |
| 12 | mg | 6 | 6 (8-12 h) | 56% | 50% |

Based on RIPA, the minimal effective dose was 2 mg and apparent saturation of the effect was achieved with the highest dose of 12 mg.
The assessment of coagulation parameters showed throughout all dose groups a decrease in Factor VIII (FVIII) and vWF levels of 20-50%, which corresponded to 14 mild adverse reactions (CTCAE grade 1) in nine subjects (eight for vWF and six for FVIII decrease)—see also Table E-2.
Table E-2: [symbol]
All other adverse events (AEs, e.g. headache and hematoma at the infusion site) occurred in the same incidence with placebo and therefore were not attributable to ALX-0081.
The only adverse drug reactions clearly attributable to ALX-0081 administration were alterations of the coagulation parameters.
No development of treatment related ALX-0081 antibodies was observed.

In conclusion, this phase I, First-in-Man Study was suitable to establish a reliable safety profile of ALX-0081 when given intravenously. ALX-0081 treatment was well tolerated and safe, no signs of bleeding were reported and no immunogenic response was detected. Mild and transient adverse events (AE) occurring in the reduction of FVIII and vWF plasma levels were observed, all AEs were fully reversible. ALX-0081 pharmacodynamic activity, measured via biomarker (RIPA), started at 2 mg and reached a maximum duration of 12 hours at 12 mg dose, inducing clinically relevant inhibition of vWF mediated platelet activation and aggregation. Non-linear pharmacokinetic properties were determined, following a 2 compartment model.

### Example 2

### Double-Blind, Placebo-Controlled, Randomized, Dose-Escalation Phase I Study to Evaluate the Safety and Efficacy of Ascending Doses of ALX-0081 in Patients with Stable Angina Undergoing Elective PCI

The study is performed mono-centric as a double-blind, placebo-controlled, randomized, dose-escalation phase I study to evaluate the safety of ascending doses of ALX-0081 (SEQ ID NO: 1) in patients with stable angina undergoing elective PCI (see Table E-1 for formulated ALX-0081 product).

### Inclusion/Exclusion Criteria:

Patients ≧18 years with stable angina (CCS≦3), undergoing elective PCI
Concomitant Aspirin. Heparin and Plavix® medication
Adequate hematological, hepatic and renal function
No previous and/or concurrent treatment with ReoPro®
No previous coronary artery bypass graft
No clinical history of DIC (Disseminated Intravascular Coagulation), thrombotic microangiopathy or coagulopathy
No clinically manifested and/or documented autoimmune cytopenia or symptomatic DIC
No severe hemorrhage≦3 months requiring blood transfusions
No stroke, TIA (transient ischemic attack) or MI (myocardial infarction)≦3 months
No chronic heart failure independent of underlying origin

The study is performed in two stages: Stage A primarily assesses tolerability whereas Stage B provides additional information on secondary endpoints. For each dose to be tested, groups of four or eight patients are randomly assigned (3:1) to receive doses of either ALX-0081 or placebo. The starting active dose in Stage A was a single dose of 2 mg ALX-0081; subsequent doses and patient numbers per dose level are presented below (see Table E-3). The start of the study drug intravenous (i.v.) infusion is 60 minutes prior to the PCI procedure. The study drug infusion is administered over 60 minutes.

Patient recruitment and treatment in the first two dose levels of Stage A followed a staggered regimen, i.e. patients are treated sequentially (i.e. one patient after another patient) with a minimum observation interval of 24 hours. Starting with dose level 3, concurrent recruitment and treatment of patients (i.e. two patients at the same time) receiving ALX-0081 and placebo is permitted in the absence of any clinically significant safety signals requiring extensive monitoring.

TABLE E-3
Dosing Schedule stage A

| | | No (%) of | No (%) of | No (%) of |
|---|---|---|---|---|
| | | subjects | subjects | subjects |
| Cohort | Treatment | randomized | exposed | completed |
| Overall | Study Drug | 12 (100%) | 12 (100%) | 12 (100%) |
| Stage A | Placebo | 4 (100%) | 4 (100%) | 4 (100%) |
| DL1 | 2 mg | 4 (100%) | 4 (100%) | 4 (100%) |
| DL2 | 4 mg | 4 (100%) | 4 (100%) | 4 (100%) |
| DL3 | 6 mg | 4 (100%) | 4 (100%) | 4 (100%) |
| DL4 | 9 mg | 4 (100%) | 4 (100%) | 4 (100%) |

### Phase Ib Stage A Preliminary Safety Summary:

vWF and FVIII levels decreased transiently as expected, but did not lead to clinical signs and symptoms (i.e. adverse events).
Absence of signs and symptoms for bleeding. Hematomas and bruises were reported due to multiple blood draws and were not associated with PCI puncture site.
The majority of reported adverse events were mild, transient and PCI procedure related.
2 SAEs occurred, both unrelated to ALX-0081, requiring prolonged hospitalization due to procedure related complications.
All adverse and serious adverse events were fully reversible.
No immunogenic responses were detected.

### Phase Ib Stage A Preliminary Pharmacodynamics Summary:

Complete RIPA inhibition is defined as decrease from baseline to <10%. Complete RICO inhibition is defined as decrease from baseline to <20%. All subjects in all dose groups achieved full RIPA/RICO inhibition at 1 h post-dosing for a maximum of 18 h/36 h.
Pronounced onset directly at the end of the infusion
High inter-individual variability
Trend indicating a dose-dependent PD effect on complement-AUC between 2 mg and 9 mg

### Phase Ib Stage A Preliminary Conclusions:

The administration of ALX-0081 in combination with Aspirin, Heparin and Plavix® was shown to be well tolerated and safe over a wide range of doses.
The observed adverse events were mild, transient and fully reversible and the majority was PCI procedure related.
ALX-0081 attributed drug effects were asymptomatic, transient and fully reversible reductions of vWF and FVIII.
RIPA and RICO were confirmed to be equivalent biomarkers indicating the biological activity of ALX-0081. The activity of ALX-0081 started at a dose level of 2 mg and reached the optimal biological level at 9 mg.
The first cohort of patients in Stage B will receive a starting dose of 6 mg, followed by 3 doses of 4 mg every 6 hours.

### Phase 1b Stage B:

In Stage B, three subsequent doses of ALX-0081 or placebo are administered every 6 hours (four doses are given in total over 24 hours) following the first dose that has been determined as safe and pharmacologically effective in Stage A (complete inhibition of vWF mediated platelet aggregation for ≧6 hours—starting dose is 6 mg, followed by 3 times 4 mg). Subsequent doses are escalated until a study drug related event occurs and/or until the target pharmacological effect (complete inhibition of vWF mediated platelet aggregation for ≧24 hours) is demonstrated. It is anticipated that up to four additional dose levels will be required in Stage B.

### Example 3

### Toxicity Studies with ALX-0081

TABLE E-4

| Study | Species | Dose | Findings |
|---|---|---|---|
| Local Tolerance | Rabbit | i.v., i.m., s.c., i.a. and Paravenous | No test item related |
| | | dose: 1.2 mg/kg | alterations |
| Single dose Toxicity | Guinea pig | Single bolus | No sign of toxicity |
| | | i.v. 2, 20 mg/kg | |
| Immunogenicity | Guinea pig | Blood samples taken from PK | No signs of |
| | | study: | immunogenicity (up |
| | | Daily dosing 700 μg/kg over 30 | to 14 days post last |
| | | days | administration) |
| PK study i.v. vs s.c. | Guinea pigs | Single bolus injection | No immunogenicity |
| | | i.v. 1, 7, 20 mg/kg | data |
| | | s.c. 1, 7, 20 mg/kg | |
| Embryo-fetal | Guinea pigs | i.m. bolus injections, once daily, | No signs of |
| development | | from 6^{th} to 41^{st} day of pregnancy | systemic maternal |
| toxicity | | 0, 0.05, 1, and 20 mg/kg | toxicity |
| | | | No test item related |
| | | | influence on |
| | | | prenatal fetal |
| | | | development |
| | | | No test item related |
| | | | malformations, |
| | | | variations or |
| | | | retardations |
| Single dose Toxicity | Cynomolgus | Single bolus | No signs of toxicity |
| | monkey | i.v. 0, 0.02, 0.4, 8 mg/kg | Dose-dependent |
| | | s.c. 0, 0.02, 0.4, 8 mg/kg | decrease of FVIII |
| | | | and vWF in |
| | | | intermediate and |
| | | | high dose group |
| | | | Signs of |
| | | | immunogenicity |
| | | | were detected in the |
| | | | highest dose groups |
| | | | (2 animals s.c. |
| | | | administered; 1 |
| | | | animal i.v. |
| | | | administered) |
| Dose-escalation | Cynomolgus | Single bolus for each escalating | goal: selection of |
| study | monkey | dose | dose levels for LPT |
| | | i.v. 0, 7.5, 74.7, 747 μg/kg | 20095/06 |
| | | Wash-out period of min. 2 days | No signs of toxicity |
| | | | No signs of |
| | | | immunogenicity (up |
| | | | to 14 days post last |
| | | | administration). |
| | | | Immunogenicity |
| | | | assessed for up to 14 |
| | | | days |
| Subchronic Toxicity | Cynomolgus | Multiple bolus injections 6 times | No sign of toxicity |
| | monkey | daily (4 hour interval) during 2 | Dose-dependent |
| | | weeks | decrease in FVIII |
| | | i.v. loading dose + first | and vWF levels in |
| | | maintenance dose + maintenance | all dose groups |
| | | dose all other days | the NOAEL was |
| | | 6 + 1.5 + 6 × 1.5 μg/kg | above 2 mg/kg b.w. |
| | | 600 + 200 + 6 × 200 μg/kg | six times daily i.v. |
| | | 6000 + 2000 + 6 × 2000 μg/kg | Signs of |
| | | | immunogenicity in |
| | | | middle and high |
| | | | dose groups during |
| | | | recovery period; No |
| | | | signs of |
| | | | immunogenicity in |
| | | | lowest dose group. |
| Subchronic Toxicity | Cynomolgus | Multiple bolus injections | No signs of toxicity |
| | monkey | 6 times daily (4 hour interval) | Dose-dependent |
| | | during 2 weeks | decrease in FVIII (in |
| | | i.v.: 0, 0.02 mg/kg | all dose groups) and |
| | | s.c.: 0, 0.02, 0.4, 2 mg/kg | vWF levels (in high |
| | | | dose group) |
| | | | the NOAEL was |
| | | | above 2 mg ALX- |
| | | | 0081/kg b.w./six |
| | | | times daily s.c. or |
| | | | i.v. |
| Immunogenicity | Baboons | Escalating doses plus 1-2 week | No signs of |
| | | intervals | immunogenicity |
| | | | identified using SPR |
| | | | or ELISA method |

The toxicity studies were conducted to establish safe testing of the compound in humans.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

All of the references described herein are incorporated by reference, in particular for the teaching that is referenced hereinabove.

1. A method for the prevention of thrombus and/or reduce the risk of thrombus formation in a patient with stable angina undergoing elective percutaneous coronary intervention (PCI) which comprises administering an effective amount of a specific A1 vWF binder to the patient with or without concomitant medication such as e.g. Heparin, acetylsalicylic acid and/or clopidogrel.

2. (canceled)

3. A method according to claim 1, wherein the specific A1 vWF binder is a compound selected from the group consisting of a polypeptide with a sequence of any of sequences SEQ ID NO: 1 to SEQ ID NO: 18, or a compound which is at least 80% identical to a sequence of any of sequences SEQ ID NO: 1 to SEQ ID NO: 18.

4. A method according to claim 1, wherein the specific A1 vWF binder is a compound selected from the group consisting of a polypeptide with a sequence of any of sequences SEQ ID NO: 1 to SEQ ID NO: 18, or a compound which is at least 80% identical to a sequence of any of sequences SEQ ID NO: 1 to SEQ ID NO: 18 and wherein the dissociation constant of any of the compounds is equal or lower than 1 nM, preferably equal or lower than 100 pM.

5. A method according to claim 1, wherein the specific A1 vWF binder is ALX-0081 (SEQ ID NO: 1).

6. A method according to claim 5, wherein ALX-0081 (SEQ ID NO: 1) is given during PCI and every 6 hours post PCI for up to 24 hours.

7. A method according to claim 5, wherein a suitable dose of ALX-0081 (SEQ ID NO: 1) is only given if the % platelet aggregation measured in ristocetin-induced platelet aggregation (RIPA) estimated for the next 6 hours is not higher than 10% or only given if the % aggregation measured in ristocetin cofactor activity (RICO) estimated for the next 6 hours is not higher than 20%, both compared to the % platelet aggregation before the administration of ALX-0081 (SEQ ID NO: 1).

8. A method according to claim 6 or 7, wherein the dose is between 2 to 12 mg, preferably 4 or 8 mg.

9. A method according to claim 1, wherein a dose of the specific A1 vWF binder is given during PCI and every 6 hours post PCI for up to 24 hours.

10. A method according to claim 9, wherein the dose of the specific A1 vWF binder is only given if the % aggregation measured in ristocetin-induced platelet aggregation (RIPA) estimated for the next 6 hours is not higher than 10% or only given if the % aggregation measured in ristocetin cofactor activity (RICO) estimated for the next 6 hours is not higher than 20%, both compared to the % platelet aggregation before the administration of the A1 vWF binder.

11. A method according to claim 9 or 10, wherein the dose is between 2 to 12 mg, preferably between 2 to 9 mg.

12. A method according to claim 1, wherein the specific A1 vWF binder comprises 12a2h1 (SEQ ID NO: 19) or a polypeptide that is at least 80% identical to SEQ ID NO: 19.

13. A method according to claim 1, wherein the specific A1 vWF binder cross-blocks at least 50% of ALX-0081 (SEQ ID NO: 1) binding and/or is cross-blocked at least 50% by ALX-0081 (SEQ ID NO: 1).

14. A method for evaluating the efficacy of a therapy using an A1 vWF binder in a patient with stable angina undergoing percutaneous coronary intervention (PCI), the method comprising:
comparing the level of platelet aggregation measured e.g. in ristocetin-induced platelet aggregation (RIPA) and/or in ristocetin cofactor activity (RICO) from the patient to a predetermined value, and
determining whether the level of platelet aggregation is at or below the predetermined level, said determination being indicative of whether the therapy is efficacious.

15. The method according to claim 14, wherein the predetermined value is 10% platelet aggregation when measured with the RIPA assay and 20% platelet aggregation when measured with the RICO assay, both compared to the % platelet aggregation with the RIPA or RICO assay respectively, before the administration of the A1 vWF binder.

16. A method of monitoring the treatment of a patient with stable angina undergoing percutaneous coronary intervention (PCI), comprising
treating a subject undergoing elective PCI with an A1 vWF binder (with or without concomitant medication such as e.g. Heparin, acetylsalicylic acid and/or clopidogrel;
obtaining blood sample from the subject; and
determining the % platelet aggregation in the sample, wherein when the % platelet aggregation after the treatment is less than the % platelet aggregation before the treatment, indicates that the subject is likely to be a responder to the therapy.

17. The method according to claim 16, wherein the % platelet aggregation after the treatment is equal or less than 10% platelet aggregation before treatment when the platelet aggregation is measured with the ristocetin-induced platelet aggregation (RIPA) assay, or is equal or less than 20% platelet aggregation before treatment when the platelet aggregation is measured with the ristocetin cofactor activity (RICO) assay.

18. A method for deciding on the course of a therapy in a human subject, comprising: (i) obtaining a level of platelet aggregation in a human subject undergoing a therapy to prevent thrombus formation and/or reduce the risk of thrombus formation, wherein the % platelet aggregation is e.g. measured by an assay selected from the group consisting of ristocetin-induced platelet aggregation (RIPA) and ristocetin cofactor activity (RICO) assays, (ii) comparing the level of platelet aggregation obtained in (i) to a predetermined value corresponding to a level of platelet aggregation in a control population (e.g. placebo group), (iii) determining whether the level of platelet aggregation obtained in (i) is equal or below the predetermined level, and (iv) deciding on the course of the therapy based on such determination.

19. The method of claim 18, wherein the predetermined value is 10% platelet aggregation if measured with the RIPA assay and 20% if measured with the RICO assay.

20. A method for preventing thrombus formation and/or reduce the risk of thrombus formation in a patient with stable angina undergoing elective percutaneous coronary intervention (PCI) with or without concomitant medication such as e.g. Heparin, acetylsalicylic acid and/or clopidogrel, the method comprising:
administering an effective amount of ALX-0081 (SEQ ID NO: 1) to a patient in need of such a treatment to lower the level of platelet aggregation in the patient below a predetermined value.

21. A method for preventing thrombus formation and/or reduce the risk of thrombus formation in a patient with stable angina undergoing elective percutaneous coronary intervention (PCI) with or without concomitant medication such as e.g. Heparin, acetylsalicylic acid and/or clopidogrel, the method comprising:
administering an effective amount of ALX-0081 (SEQ ID NO: 1) to a subject in need of such a prevention,
detecting a level of platelet aggregation in the patient undergoing a therapy,
comparing the level of platelet aggregation to a predetermined value, and optionally
administering a second and/or further effective amount of ALX-0081 (SEQ ID NO: 1) to a patient based on the level of the platelet aggregation.

22. A method for identifying a patient disposed to respond favorably to ALX-0081 (SEQ ID NO: 1), which method comprises detecting % platelet aggregation in a blood sample from the patient and treating the patient with an effective amount of ALX-0081 (SEQ ID NO: 1), wherein the % platelet aggregation in the blood sample from the patient is 10% when measured in the ristocetin-induced platelet aggregation (RIPA) assay and 20% when measured in the ristocetin cofactor activity (RICO) assay.

23. (canceled)


SEQUENCE LISTING (19 sequences)

SEQ ID NO: 1 - protein, 259 aa, Artificial
  Nanobody and/or Nanobody fragment
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSSAAAEVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSS

SEQ ID NO: 2 - protein, 259 aa, Artificial
  Nanobody and/or Nanobody fragment
QVKLEESGGGLVQAGGALRLSCAASGRTFSYNPMGWFRQAPGKERDLVAAISRTGGSTYYPDSVEGRFTISRDNAKRMVYLQMNNLKPEDTAVYYCAAAGVRAEDGRVRTLPSEYTFWGQGTQVTVSSAAAEVQLVESGGGLVQAGGALRLSCAASGRTFSYNPMGWFRQAPGKERDLVAAISRTGGSTYYPDSVEGRFTISRDNAKRMVYLQMNNLKPEDTAVYYCAAAGVRAEDGRVRTLPSEYTFWGQGTQVTVSS

SEQ ID NO: 3 - protein, 265 aa, Artificial
  Nanobody and/or Nanobody fragment
QVKLEESGGGLVQAGGALRLSCAASGRTFSYNPMGWFRQAPGKERDLVAAISRTGGSTYYPDSVEGRFTISRDNAKRMVYLQMNNLKPEDTAVYYCAAAGVRAEDGRVRTLPSEYTFWGQGTQVTVSSGGGGSGGGSEVQLVESGGGLVQAGGALRLSCAASGRTFSYNPMGWFRQAPGKERDLVAAISRTGGSTYYPDSVEGRFTISRDNAKRMVYLQMNNLKPEDTAVYYCAAAGVRAEDGRVRTLPSEYTFWGQGTQVTVSS

SEQ ID NO: 4 - protein, 286 aa, Artificial
  Nanobody and/or Nanobody fragment
QVKLEESGGGLVQAGGALRLSCAASGRTFSYNPMGWFRQAPGKERDLVAAISRTGGSTYYPDSVEGRFTISRDNAKRMVYLQMNNLKPEDTAVYYCAAAGVRAEDGRVRTLPSEYTFWGQGTQVTVSSGGGGSGGGGSGGGGSGGGGSGGGGSGGGGSEVQLVESGGGLVQAGGALRLSCAASGRTFSYNPMGWFRQAPGKERDLVAAISRTGGSTYYPDSVEGRFTISRDNAKRMVYLQMNNLKPEGTAVYYCAAAGVRAEDGRVRTLPSEYTFWGQGTQVTVSS

SEQ ID NO: 5 - protein, 247 aa, Artificial
  Nanobody and/or Nanobody fragment
AVQLVESGGGLVQPGGSLRLSCLASGRIFSIGAMGMYRQAPGKQRELVATITSGGSTNYADPVKGRFTISRDGPKNTVYLQMNSLKPEDTAVYYCYANLKQGSYGYRFNDYWGQGTQVTVSSAAAEVQLVESGGGLVQPGGSLRLSCLASGRIFSIGAMGMYRQAPGKQRELVATITSGGSTNYADPVKGRFTISRDGPKNTVYLQMNSLKPEDTAVYYCYANLKQGSYGYRFNDYWGQGTQVTVSS

SEQ ID NO: 6 - protein, 253 aa, Artificial
  Nanobody and/or Nanobody fragment
AVQLVESGGGLVQPGGSLRLSCLASGRIFSIGAMGMYRQAPGKQRELVATITSGGSTNYADPVKGRFTISRDGPKNTVYLQMNSLKPEDTAVYYCYANLKQGSYGYRFNDYWGQGTQVTVSSGGGGSGGGSEVQLVESGGGLVQPGGSLRLSCLASGRIFSIGAMGMYRQAPGKQRELVATITSGGSTNYADPVKGRFTISRDGPKNTVYLQMNSLKPEDTAVYYCYANLKQGSYGYRFNDYWGQGTQVTVSS

SEQ ID NO: 7 - protein, 274 aa, Artificial
  Nanobody and/or Nanobody fragment
AVQLVESGGGLVQPGGSLRLSCLASGRIFSIGAMGMYRQAPGKQRELVATITSGGSTNYADPVKGRFTISRDGPKNTVYLQMNSLKPEDTAVYYCYANLKQGSYGYRFNDYWGQGTQVTVSSGGGGSGGGGSGGGGSGGGGSGGGGSGGGGSEVQLVESGGGLVQPGGSLRLSCLASGRIFSIGAMGMYRQAPGKQRELVATITSGGSTNYADPVKGRFTISRDGPKNTVYLQMNSLKPEDTAVYYCYANLKQGSYGYRFNDYWGQGTQVTVSS

SEQ ID NO: 8 - protein, 259 aa, Artificial
  Nanobody and/or Nanobody fragment
QVQLVESGGGLVQAGGALRLSCAASGRTFSYNPMGWFRQAPGKERDVVAAISRTGGSTYYARSVEGRFTISRDNAKRMVYLQMNALKPEDTAVYYCAAAGVRAEDGRVRTLPSEYNFWGQGTQVTVSSAAAEVQLVESGGGLVQAGGALRLSCAASGRTFSYNPMGWFRQAPGKERDVVAAISRTGGSTYYARSVEGRFTISRDNAKRMVYLQMNALKPEDTAVYYCAAAGVRAEDGRVRTLPSEYNFWGQGTQVTVSS

SEQ ID NO: 9 - protein, 265 aa, Artificial
  Nanobody and/or Nanobody fragment
QVQLVESGGGLVQAGGALRLSCAASGRTFSYNPMGWFRQAPGKERDVVAAISRTGGSTYYARSVEGRFTISRDNAKRMVYLQMNALKPEDTAVYYCAAAGVRAEDGRVRTLPSEYNFWGQGTQVTVSSGGGGSGGGSEVQLVESGGGLVQAGGALRLSCAASGRTFSYNPMGWFRQAPGKERDVVAAISRTGGSTYYARSVEGRFTISRDNAKRMVYLQMNALKPEDTAVYYCAAAGVRAEDGRVRTLPSEYNFWGQGTQVTVSS

SEQ ID NO: 10 - protein, 286 aa, Artificial
  Nanobody and/or Nanobody fragment
QVQLVESGGGLVQAGGALRLSCAASGRTFSYNPMGWFRQAPGKERDVVAAISRTGGSTYYARSVEGRFTISRDNAKRMVYLQMNALKPEDTAVYYCAAAGVRAEDGRVRTLPSEYNFWGQGTQVTVSSGGGGSGGGGSGGGGSGGGGSGGGGSGGGGSEVQLVESGGGLVQAGGALRLSCAASGRTFSYNPMGWFRQAPGKERDVVAAISRTGGSTYYARSVEGRFTISRDNAKRMVYLQMNALKPEDTAVYYCAAAGVRAEDGRVRTLPSEYNFWGQGTQVTVSS

SEQ ID NO: 11 - protein, 259 aa, Artificial
  Nanobody and/or Nanobody fragment
EVQLVESGGGLVQPGGSLRLSCAASGRTFSYNPMGWFRQAPGKGRELVAAISRTGGSTYYPDSVEGRFTISRDNAKRSVYLQMNSLRAEDTAVYYCAAAGVRAEDGRVRTLPSEYTFWGQGTQVTVSSAAAEVQLVESGGGLVQPGGSLRLSCAASGRTFSYNPMGWFRQAPGKGRELVAAISRTGGSTYYPDSVEGRFTISRDNAKRSVYLQMNSLRAEDTAVYYCAAAGVRAEDGRVRTLPSEYTFWGQGTQVTVSS

SEQ ID NO: 12 - protein, 259 aa, Artificial
  Nanobody and/or Nanobody fragment
EVQLVESGGGLVQPGGSLRLSCAASGRTFSYNPMGWFRQAPGKGREVVAAISRTGGSTYYARSVEGRFTISRDNAKRMVYLQMNSLRAEDTAVYYCAAAGVRAEDGRVRTLPSEYNFWGQGTQVTVSSAAAEVQLVESGGGLVQPGGSLRLSCAASGRTFSYNPMGWFRQAPGKGREVVAAISRTGGSTYYARSVEGRFTISRDNAKRMVYLQMNSLRAEDTAVYYCAAAGVRAEDGRVRTLPSEYNFWGQGTQVTVSS

SEQ ID NO: 13 - protein, 265 aa, Artificial
  Nanobody and/or Nanobody fragment
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSSGGGGSGGGSEVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSS

SEQ ID NO: 14 - protein, 265 aa, Artificial
  Nanobody and/or Nanobody fragment
EVQLVESGGGLVQPGGSLRLSCAASGRTFSYNPMGWFRQAPGKGRELVAAISRTGGSTYYPDSVEGRFTISRDNAKRSVYLQMNSLRAEDTAVYYCAAAGVRAEDGRVRTLPSEYTFWGQGTQVTVSSGGGGSGGGSEVQLVESGGGLVQPGGSLRLSCAASGRTFSYNPMGWFRQAPGKGRELVAAISRTGGSTYYPDSVEGRFTISRDNAKRSVYLQMNSLRAEDTAVYYCAAAGVRAEDGRVRTLPSEYTFWGQGTQVTVSS

SEQ ID NO: 15 - protein, 265 aa, Artificial
  Nanobody and/or Nanobody fragment
EVQLVESGGGLVQPGGSLRLSCAASGRTFSYNPMGWFRQAPGKGREVVAAISRTGGSTYYARSVEGRFTISRDNAKRMVYLQMNSLRAEDTAVYYCAAAGVRAEDGRVRTLPSEYNFWGQGTQVTVSSGGGGSGGGSEVQLVESGGGLVQPGGSLRLSCAASGRTFSYNPMGWFRQAPGKGREVVAAISRTGGSTYYARSVEGRFTISRDNAKRMVYLQMNSLRAEDTAVYYCAAAGVRAEDGRVRTLPSEYNFWGQGTQVTVSS

SEQ ID NO: 16 - protein, 286 aa, Artificial
  Nanobody and/or Nanobody fragment
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSSGGGGSGGGGSGGGGSGGGGSGGGGSGGGGSEVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSS

SEQ ID NO: 17 - protein, 286 aa, Artificial
  Nanobody and/or Nanobody fragment
EVQLVESGGGLVQPGGSLRLSCAASGRTFSYNPMGWFRQAPGKGRELVAAISRTGGSTYYPDSVEGRFTISRDNAKRSVYLQMNSLRAEDTAVYYCAAAGVRAEDGRVRTLPSEYTFWGQGTQVTVSSGGGGSGGGGSGGGGSGGGGSGGGGSGGGGSEVQLVESGGGLVQPGGSLRLSCAASGRTFSYNPMGWFRQAPGKGRELVAAISRTGGSTYYPDSVEGRFTISRDNAKRSVYLQMNSLRAEDTAVYYCAAAGVRAEDGRVRTLPSEYTFWGQGTQVTVSS

SEQ ID NO: 18 - protein, 286 aa, Artificial
  Nanobody and/or Nanobody fragment
EVQLVESGGGLVQPGGSLRLSCAASGRTFSYNPMGWFRQAPGKGREVVAAISRTGGSTYYARSVEGRFTISRDNAKRMVYLQMNSLRAEDTAVYYCAAAGVRAEDGRVRTLPSEYNFWGQGTQVTVSSGGGGSGGGGSGGGGSGGGGSGGGGSGGGGSEVQLVESGGGLVQPGGSLRLSCAASGRTFSYNPMGWFRQAPGKGREVVAAISRTGGSTYYARSVEGRFTISRDNAKRMVYLQMNSLRAEDTAVYYCAAAGVRAEDGRVRTLPSEYNFWGQGTQVTVSS

SEQ ID NO: 19 - protein, 128 aa, Artificial
  Nanobody and/or Nanobody fragment
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSS